(19) United States
(12) Patent Application Publication
(10) Pub. No.: US 2014/0148847 A1
(43) Pub. Date: May 29, 2014

(54) DEVICE FOR SEALING PERFORATIONS IN BLOOD VESSELS

(71) Applicant: DELIVERANCE, Katzerin (IL)

(72) Inventor: Dan Aravot, Haifa (IL)

(73) Assignee: DELIVERANCE, Katzerin (IL)

(21) Appl. No.: 14/168,462

(22) Filed: Jan. 30, 2014

Related U.S. Application Data
(63) Continuation-in-part of application No. PCT/IL2013/050207, filed on Mar. 7, 2013.
(60) Provisional application No. 61/608,127, filed on Mar. 8, 2012.

(51) Int. Cl. A61B 17/00

(52) U.S. Cl. CPC A61B 17/0057
USPC 606/213

(57) ABSTRACT

The present invention relates to a device and methods of using the device for dealing with perforations in tissue, such as blood vessels and organs. The device includes a body having an inner member and an outer member slidable over the inner member, a distal member operably coupled to the inner member, wherein the distal member has a retracted and a deployed configuration. The device further includes a proximal member operably coupled to the outer member and has a retracted configuration in which the proximal member is substantially flat against the body and a deployed configuration. The device may be used in traumatic medical practices, either in the field, as well as in emergency and/or operating rooms. In particular, the device may be used in planned general and/or minimally invasive operations, such as during laparoscopic, thoracoscopic, and other minimally invasive surgeries that may have caused iatrogenic major vascular injury.

### RELATED APPLICATIONS

The present application a continuation-in-part of PCT international patent application number PCT/IL2013/050207, filed Mar. 7, 2013, which claims the benefit of and priority to U.S. provisional patent application Ser. No. 61/608,127, filed Mar. 8, 2012, the content of each of which is incorporated by reference herein in its entirety.

### TECHNICAL FIELD

The present invention is generally related to a medical device or tool, and methods of use of such device for plugging or closing apertures in blood vessels, heart or other organs. The device may be used in traumatic medical practices, either in the field for services provided by emergency medical services, as well as in emergency and/or operating rooms. In particular, the device may be used in planned general and/or minimally invasive operations, such as during laparoscopic, thoracoscopic, and other minimally invasive surgeries that may have caused iatrogenic major vascular injury.

### BACKGROUND

A main causes of death in trauma injuries is hemorrhage (i.e., bleeding) from the circulatory system, such as from major blood vessels of the torso or the heart. If not efficiently treated without delay, the hemorrhage causes exsanguination and may lead to eventual death. Depending upon age, health, and fitness level of an individual, a person can die from losing half or two-thirds of their blood, and a loss of roughly one-third of the blood volume is considered very serious. Exsanguination is the estimated cause of death in 5-10% of urban trauma injuries and 10% or more of military trauma injuries. Accordingly, procedures to repair trauma to blood vessels or organs must generally be performed as quickly as possible so as to minimize exsanguination.

For example, laparoscopic and minimally invasive surgeries have become increasing popular due in large part to their convenience as well as proven safety. However, major vascular injuries during laparoscopic and minimally invasive surgeries can have devastating effects.

Some studies reported a 0.22 to 1.1% rate of incidence in laparoscopic surgery, resulting in an associated mortality rate of 8 to 17%. Patients undergoing complex procedures, especially those involving dissection near major vascular structures and inflammatory processes, generally have a higher rate of vascular injury.

Devices and methods exist for stopping hemorrhaging by sealing a perforation within tissue, such as a perforation in a blood vessel. A problem with certain of those devices is that they operate by sealing only one side of a perforation. For example, some devices seal only an inside of a perforation. Other devices seal only an outside of a perforation. Sealing from only the inside may result in incomplete sealing of the perforation. Sealing from only the outside requires application of direct or radial pressure to an exterior of the vessel, which may result in a complete cave-in of the vessel or obstruction of blood flow.

Occluders have been used to entirely block blood flow to a perforation until it can be repaired. The use of occluders, however, often requires complete, or near-complete, exposure of blood vessels from surrounding tissues so as to enable efficient blocking, which is rarely the case in iatrogenic major vascular injury. Another problem with occluders is that entirely blocking flow within a vessel may result in ischemia or a stroke, which can lead to death.

### SUMMARY

The invention generally provides low profile devices that permit rapid sealing from both an inside and an outside of a perforation in a tissue without completely blocking flow within the tissue. In that manner, devices of the invention maintain blood flow to tissue while completely sealing a perforation without causing complete cave-in of the tissue.

In certain aspects, devices of the invention include a body having an inner member and an outer member that is slidable over the inner member. A distal member is operably coupled to the inner member and has a retracted and a deployed configuration. A proximal member is operably coupled to the outer member and also has a retracted configuration in which the proximal member is substantially flat against the body and a deployed configuration in which a portion of the proximal member expands away from the body. The distal and proximal members can be operated independently of each other such that the distal member can be deployed on one side of a perforation prior to deployment of the proximal member. Subsequently, the proximal member can be deployed and slid into position on the other side of the perforation, thereby sealing it. The flexible distal portion of the device and the streamline distal and proximal members allow for an operator to easily gain access to an injury site. The independent operation of the distal and proximal members and the slideability of the proximal member provides for simple operation of the device to rapidly and easily gain control the hemorrhaging.

The inner and outer members may take numerous different forms. Typically, the inner and outer members are in the form of tubular structures. The device may also include, for example, a handheld actuation console coupled to the inner and outer members for transforming the distal and proximal members between the retracted and deployed configurations.

In operation, once a surgeon, or other medical personnel (e.g., first responder), has gained access to an injured blood vessel, the device may be inserted such that the distal member and a portion of the inner member passes through the perforation and into a lumen of the blood vessel.

Typically during insertion, although not required, the distal member is in the retracted configuration, so as to allow the distal member to easily pass through the perforation and be received within the lumen of the blood vessel. The distal member is then transformed from the retracted configuration to the deployed configuration so as to block the perforation from an inner side of the vessel. In one embodiment, retraction of the inner member may cause the distal member to compress and radially expand from the retracted configuration to the deployed configuration, thereby forming a discoid-type shape and blocking the perforation from within the blood vessel.

The proximal member may then be positioned adjacent to the perforation on an outer side of the blood vessel. For example, in one embodiment, sliding of the outer member relative to the inner member and in a direction towards the distal member results in movement of the proximal member towards the perforation. Once in a desired position, the proximal member is transformed from the retracted configuration to the deployed configuration so as a block the perforation from the outer side of the vessel. In one embodiment, the proximal member may be composed of an expandable material and may be in the form of a balloon. Upon transformation of the proximal member to the deployed configuration, a tight seal of the perforation is generally formed between the distal and proximal members from both inside and outside of the vessel's wall around the perforation, thereby providing control of the hemorrhaging without obstructing total blood flow within the vessel.

Upon sealing the perforation, the damaged blood vessel may then be repaired laparoscopically or by the open technique in a controlled and safer environment. For example, temporary fasteners may be positioned on either side of the sealed perforation so as to temporarily prevent the flow of fluid in between. The proximal member may then be transformed from the deployed to the retracted configuration and pulled away from the vessel. The distal member may also be transformed from the deployed to the retracted configuration and removed from within the repaired vessel. Upon removal of the device, the perforation can then be repaired (e.g., by way of sutures, staples, etc.) and the temporary fasteners removed to restore flow through the repaired vessel. In one embodiment, the distal member is bioresorbable and may be detachable from the inner member, such that the distal member may remain within the vessel during repair of the perforation and be released within the blood vessel while the remainder of the device may be removed from the trauma site.

In certain embodiments, the device of the present invention is configured to allow fluid to continue to flow within the blood vessel, as well as allow exchange of fluid to or from the blood vessel via the sealed perforation. For example, the inner member of the device may further have a lumen through which fluid may pass there through. For example, while the distal and proximal members are in deployed configurations and the perforation is sealed, fluids may be allowed to pass into the blood vessel via the lumen of the inner member. Accordingly, organs or other tissues supplied by blood from the damaged vessel are not starved of blood (ischemia), which would otherwise result in serious consequences such as organ damage, or mortality.

Additionally, prior to deployment of the distal and/or proximal members and sealing of the perforation, the lumen may be used to pump in air or liquid to clear away obstructing material from the locus of operation. Additionally, or alternatively, the lumen may be used for the removal of fluids or obstructing debris from the perforation site or locus of operation. For example, the lumen can be used to suck in liquids from the locus of operation, usually for clearing obstructing fluids, such as excess blood. The suction action can be achieved by connecting the inner member to a vacuum source, such as a vacuum pump.

### BRIEF DESCRIPTION OF THE DRAWINGS

FIGS. 1A-D are isometric views of the distal end of the probe of the tool of the invention.

FIG. 2 is an isometric view of the tip of the probe of the tool of the invention.

FIGS. 3A-B are isometric views of the association of the balloon with the probe.

FIGS. 3C-D are isometric views of the deployment aspects relating to the balloon and discoid.

FIG. 4 is an isometric view of the pumping mechanism for inflating the balloon.

FIGS. 5A-C are isometric views of the internal components of the console and probe in steps of the deployment of the probe.

FIGS. 6A-B are isometric views of the internal components of the console showing two spring types deployed in one functional device.

### DETAILED DESCRIPTION

The present invention is a medical device or tool with an aim of plugging or closing apertures in blood vessels, heart or other organs. The device may be used in traumatic medical practices, either in the field, such as those provided by emergency medical services (EMS), as well as in the emergency and/or operating rooms. In particular, the device may be used in planned general and/or minimally invasive operations, such as during laparoscopic, thoracoscopic, and other minimally invasive surgeries (MIS) that may have caused iatrogenic major vascular injury.

A typical tool in accordance with the invention includes an actuation console and a probe. The tool is utilizable in medical practices such as sealing traumatic perforations or planned incision in blood vessels, heart and other tissues.

### The Probe

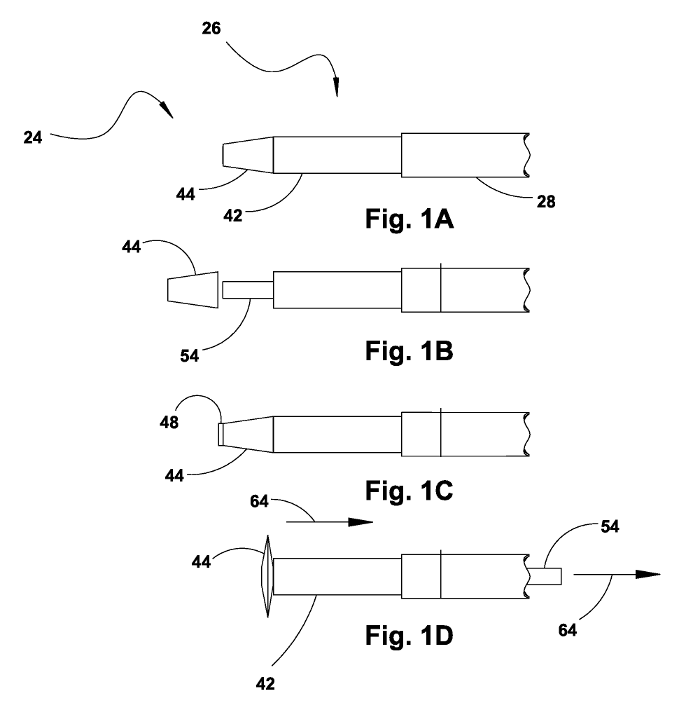

In FIGS. 1A-1D some details of the probe of the tool of the invention are shown. The distal end 24 of the probe 26 is the end that contacts the body of the treated individual. Enveloping tube 28 is a longitudinal cylindrical tube that harbors in its lumen several other longitudinal components of the system as will be described below, the distal portion of it is shown. Intermediate tube 42 is coaxial with enveloping tube 28 and somewhat smaller in diameter, at the distal end of which sleeve 44 is located, tapering towards the end. In FIG. 1B at the distal end of the probe, sleeve 44 is shown removed from its operational location, exposing inner cylindrical tube 54. Inner tube 54 is coaxial with enveloping tube 28. As shown in FIG. 1C, in one embodiment, at the extreme end of inner tube 54 retracting ring 48 is connected. In embodiments in which retracting ring 48 is absent, the tapering tip of sleeve 44 is glued or otherwise firmly attached to the end of inner tube 54. In FIG. 1D, sleeve 44 is shown compressed, as a result of the compression effected by inner tube 54 in the direction of arrow 64, forming a discoid bulge and reaching as far as the distal end of intermediate cylindrical tube 42.

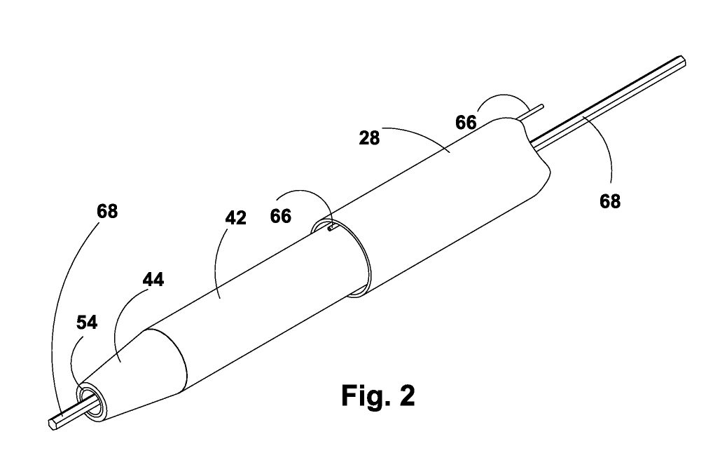

At FIG. 2, a closer view of the tip of the probe of the invention is shown, at the distal end (the side proximal to the patient), sleeve 44 is substantially flush with the end of inner tube 54.

Guide wire 68 is an optional component that runs through the entire length of the probe, in the lumen of inner tube 54 and is extendable out said lumen distally. A role of the guide wire is to help insert the distal end of the probe inside a rupture in a vessel and ruptures/perforations in other organs of the body. Balloon feed tube (BFT) 66 runs from the console in the lumen of enveloping tube 28 is not coaxial with the probe, its function will be explained below.

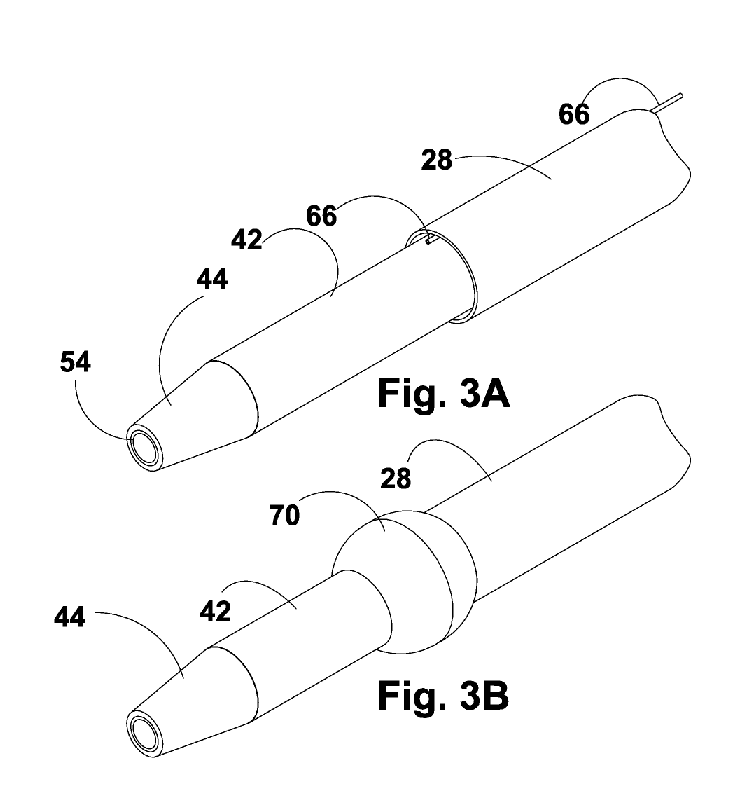

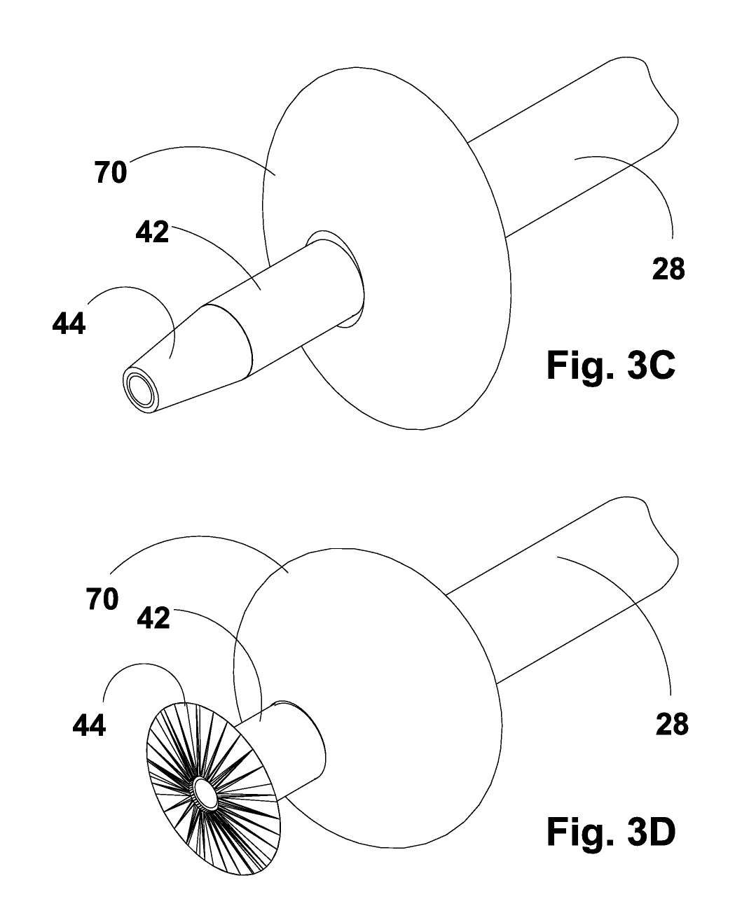

In FIG. 3A the distal end of the probe is shown, with sleeve 44, the face of inner tube 54, intermediate tube 42, BFT 66 and enveloping tube 28. In FIG. 3B, balloon 70 is shown, somewhat inflated. The orifice, not shown here, of BFT 66 opens into the lumen of balloon 70. It is noted at this point that the balloon in firmly attached to the distal end of enveloping tube 28, or even best described as being an integral part of the distal part of tube 28. The balloon is slidable, either touching or non-touching on intermediate tube 42. In FIGS. 3C-3D, balloon 70 is shown assuming two somewhat different shapes, in FIG. 3C the balloon appears as having a toroidal shape and in FIG. 3D the balloon is still toroidal but is more spherically shaped.

In FIG. 3D, the two shape shifting modules in the tool of the invention are deployed, namely conical sleeve 44 is compressible to form a discoid, and concomitantly or subsequently balloon 70 is inflated causing tight seal of vessel's rupture/perforation from both inner and outer sides. In FIG. 3B, BFT 66 is the tube that reaches the lumen of balloon 70 to facilitate the inflation of the balloon from the user side. To summarize major properties of the tubes that make up the probe, all three are coaxial and even diameter all along, featuring free tubular space in between adjacent tubes, and a cylindrical lumen in the axial zone.

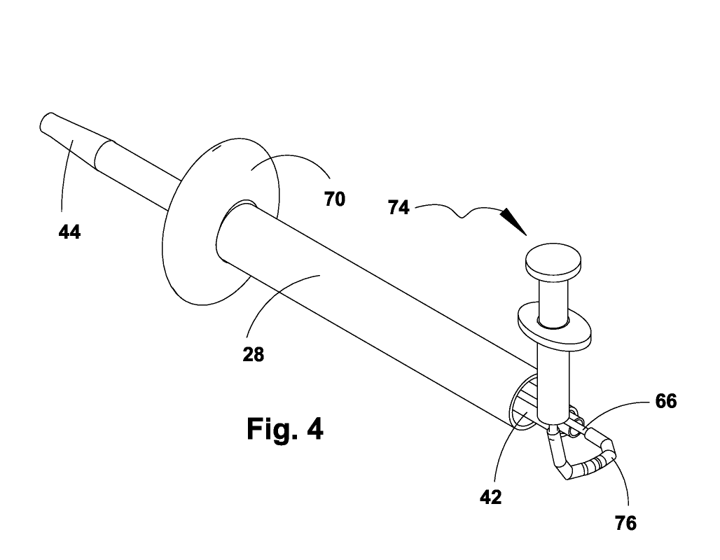

As shown in FIG. 4, a syringe 74 having a spout is connected to tubing 76 which is a functional continuation of tubing 66. By pressing syringe 74, air, gas or a suitable liquid is passed from the syringe to balloon 70 inflating it if the force required to do so is obtained by the pressure applied by the syringe. The syringe can be activated manually or by applying mechanical, electric or hydraulic actuators. The syringe is a simple device readily available, but any suitable pump is applicable for use as a balloon inflating device in this tool.

### Deployment of the Probe and Structural Aspects

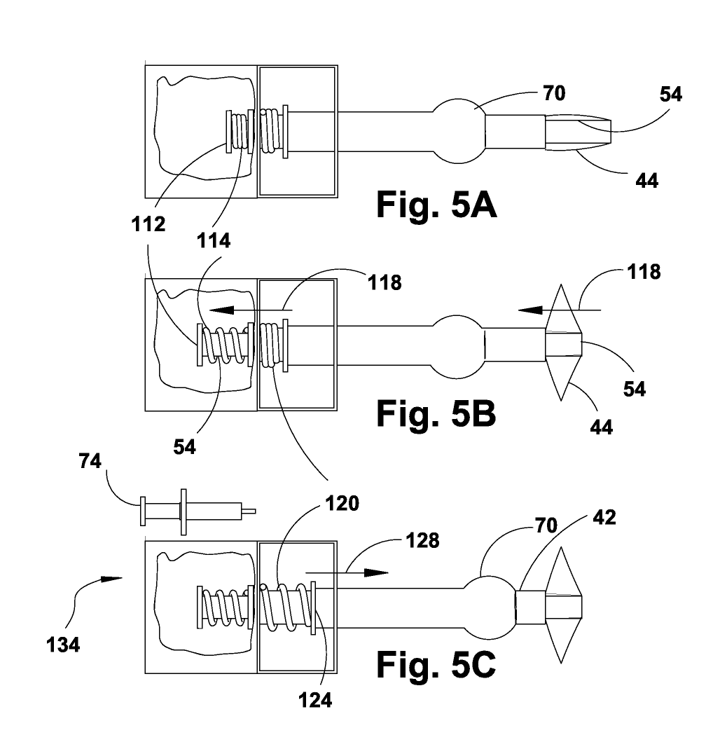

As described herein, the sleeve is retracted and forced into a discoid by the movement of the inner tube towards the proximal direction, towards the user. This step is however typically reversible and the inner tube can be pushed forwards again, with a subsequent quenching of the discoid re-forming a sleeve. Typically, a full deployment of the probe of the invention is a procedure involving a sequence of three steps. In a first step (step one), the sleeve is contracted forming a sealed discoid stopper for permitting sealing of a perforation in a vessel or other organ from the inner side. In the next step (step two), the deflated balloon is slid on or about the surface of the intermediate tube towards the discoid, and in a subsequent step (step three), the balloon is inflated providing close and tight sealing of the perforation in the blood vessel or other organ, by securing the perforation from the inner as well as the outer sides. More pictorial explanations of the deployment procedure are presented in FIGS. 5A-5C.

In FIG. 5A, collar 112 of the inner tube 54 holds a contracted spring 114. Balloon 70 is deflated and sleeve 44 is quenched. At this state the probe can be inserted into a perforation or a vessel. Once the tip has been inserted into a perforation, for example, spring 114 is released by pressing a trigger (not shown). Collar 112 is urged by spring 114 to move in the direction of arrow 118. As a result inner tube 54 moves towards the console and sleeve 44 compresses against the distal end of the intermediate tube lengthwise (in the direction of the probe) and spreads sideways, forming a vertically disposed discoid. At this state discoid 44 can practically be set against a perforation in a blood vessel. At this stage, spring 120 is contracted.

As shown in FIG. 5C, collar 124 is released, spring 120 expands horizontally in the direction of arrow 128, pushing collar 124 and the attached enveloping tube 28 with it in the direction of arrow 128, pushing balloon 70 in the same direction, sliding over or touching intermediate tubing 42. Therefore intermediate tube remains static with respect to console 134 throughout the deployment. At this stage, step three of the deployment is realized, by activating pump 74 to inflate balloon 70 (in this drawing shown quenched). As a general comment for these drawings (FIGS. 5A-5C), the probe could be also much longer in reality with respect to the console for example for the use in laparoscopic and thoracoscopic and endoscopic procedures. For the sake of convenience it was drawn as if the probe is much shorter.

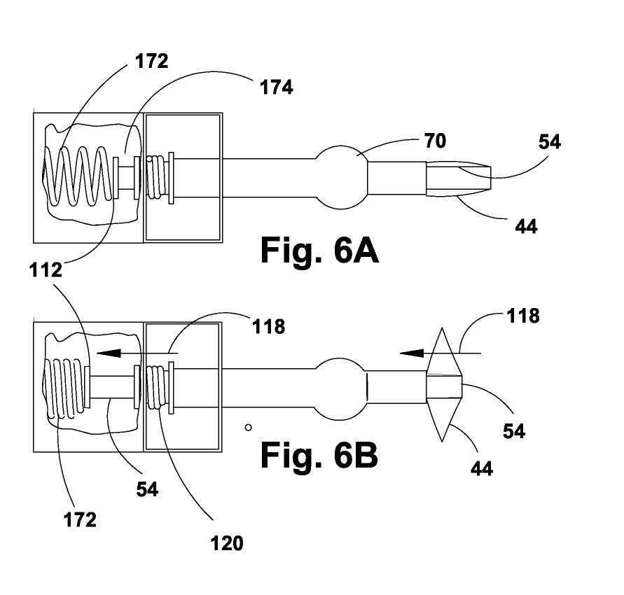

In the ongoing description of FIGS. 5A-5C the properties of the springs lends themselves readily to store kinetic energy when they are compressed, for example in FIG. 5C both springs are shown compressed, ready to deliver their kinetic energy when released. In other embodiments, if other type of spring is used, for example tension spring in which the kinetic energy is stored when the spring is stretched rather than contracted, the arrangement of the spring(s) is different as can be seen in FIGS. 6A-6B. In FIG. 6A, spring 114 is absent, leaving an empty space 174 and instead, spring 172 is to be seen deployed to the left of collar 112 (compared with FIG. 5A). In FIG. 6A, spring is stretched, having stored potential energy that is delivered as kinetic energy when the spring is released. A released form of coil 172 is seen in FIG. 6B (compared with FIG. 5B). The same principle can be applied to the other spring in the system or to both springs. Moreover, instead of springs, elastic structures can be used as kinetic energy storing element other than springs, such as rubber structures, or plastic resin structures.

Another possibility is to use pneumatic (“gas”) struts which employ compression of gas as kinetic energy source. Indeed any kinetic energy storing element can be used to drive the tubes as described above providing they can move the mechanical structures in the geometrical constraints as dictated by the structural specifications.

### Materials and Additional Properties of the Tool of the Invention

The sleeve, or discoid, of the invention are preferably made from braided medical fiber, as known in the art. Typically, the braided fibers forming a mesh of some sort, are impregnated with an impervious material such as a resin or rubber, as known in the art. Otherwise, the braided fibers can be completely covered by a plastic resin extruded cap that fits the area of the discoid. Additionally for some uses, the sleeve together with impregnated material are made of biodegradable materials (such as polyglycolic acid fiber) in order to facilitate leaving the sleeve inside the body for specific uses and allow for its disappearance by gradual absorption in the body. In such cases as the sleeve is to be left inside the patient, it may become necessary to engage the sleeve more strongly into the tissues of the patient, such as by suturing or gluing. The sleeve should not necessarily form a discoid upon contracting, other forms can be designed to suit different type of openings in the vessels. In a similar line of versatility, instead of a balloon at the distal end of enveloping tube 28, a shape shifting element rather in the form of sleeve 44 may be employed. Mechanically it may function without the need for a pump but rather be in a semi-stable state that when activated it would promptly change shape from a cylindrical/conical state to a spread out state, very similar to an umbrella.

### Some Mechanical Aspects of the Probe

Optimal flexibility of the probe is a parameter that depends on the intended use of the probe. For example for endoscopic and laparoscopic uses greater flexibility at the distal part of the probe is advantageous than as required for other trauma treatments. In one aspect of the invention, the distal part of the probe is more flexible than the proximal part. This distribution of mechanical properties along the length of the probe allows the probe to be pushed forcefully forwards, yet at the distal part, some flexibility is bestowed, for example for approaching different perforation sites from most suited angle by fine tuning the placement of the tip by another tool, without maneuvering of the entire probe.

### Deployment Scenarios

In one scenario, the probe is pushed through a perforation in a blood vessel. When the tip is definitely inserted inside the vessel, the first step is invoked in which the discoid is formed from the sleeve by applying compression on the sleeve. Upon compression of the sleeve and subsequent formation of the discoid, a protective structure is formed within the vessel. A user of the probe, such as a surgeon, medical personnel or the like, may then position the discoid such that it makes contact with the inner side of the rupture and a portion of the surrounding inner wall of the vessel, thereby blocking the perforation from within. For example, the user may slightly pull the probe away from the vessel so as to bring the discoid into direct contact with the perforation. Then, the balloon is brought up to proximity of the vessel by invoking the next step (step two). Then, at step three, the balloon is inflated closing in on the perforation. The discoid is flattened, the punctured vessel is sutured, and the discoid released from the tip completely and the balloon and the rest of the probe pulled back. In another scenario, while the discoid—balloon couple is stopping the perforation, two temporary endoscopic vessel clips, or tightening bands are applied to the damaged vessel each on a respective side of the damaged vessel, stopping the blood transport in the vessel. The clips are such as offered under the code PL522R, by Aesculap Inc, of 3773 Corporate Parkway Center Valley, Pa. 18034, USA. Instead of clips, constricting bands may be used. When this is accomplished, the discoid is quenched by pushing the inner tube forwards (in the direction away from the console), and the tip can be removed from the now dry vessel. After which the perforation can be sutured, and subsequently compressing on both sides released. In another scenario, when the probe of the invention is fully deployed the vessel is sutured with the discoid inside, after which the discoid is detached from the probe without reverting to a flattened quenched position.

The lumen of the inner tube of the probe can be used to pump in air or liquid to clear away obstructing material from the locus of operation, which may generally improve the overall view of the trauma site, allowing improved identification of the perforation site and subsequent introduction of the of distal portion of the probe. The lumen can be also used to introduce fluid into the blood vessels during surgery. Reversely, the same lumen can be used to suck in liquids from the locus of operation, usually for clearing obstructing fluids, usually blood. The suction action can be achieved by connecting the inner tube at its proximal end to a vacuum pump or to an existing suction machine using an adaptor. Nevertheless, fluids can be delivered to the locus of intervention rather than sucked to achieve clearing of debris or obstructing matter, saline is an obvious candidate fluid in such cases.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A device for sealing a perforation in a tissue, the device comprising:
a body comprising an inner member and an outer member that is slidable over the inner member;
a distal member operably coupled to the inner member, the distal member comprising a retracted and a deployed configuration; and
a proximal member operably coupled to the outer member, the proximal member comprising a retracted configuration in which the proximal member is substantially flat against the body and a deployed configuration.

2. The device of claim 1, wherein the body further comprises an intermediate member, a portion of which surrounds the inner member.

3. The device of claim 2, wherein the outer member is slidable over the intermediate member.

4. The device of claim 3, wherein the inner, the intermediate, and the outer members are coaxially positioned about each other.

5. The device of claim 4, wherein the proximal member is a balloon.

6. The device of claim 5, wherein retraction of the inner member causes the distal member to compress against the intermediate member, thereby causing compression and radial expansion of the distal member from the retracted configuration to the deployed configuration.

7. The device of claim 1, wherein the distal member is detachable from the body.

8. The device of claim 5, further comprising a pump that is operably coupled to the balloon.

9. The device of claim 1, wherein the distal member comprises a braided fiber sleeve.

10. The device of claim 1, wherein the distal member is composed of a biodegradable material.

11. The device of claim 1, wherein the device is configured such that liquid is prevented from entering the device.

12. A method for sealing a perforation in a tissue, the method comprising:
providing a device that comprises a body comprising an inner member and an outer member that is slidable over the inner member; a distal member operably coupled to the inner member, the distal member comprising a retracted and a deployed configuration; and a proximal member operably coupled to the outer member, the proximal member comprising a retracted configuration in which the proximal member is substantially flat against the body and a deployed configuration.
inserting the device such that the inner member comprising the distal member is through a perforation in a tissue;
transforming the distal member from the retracted configuration to the deployed configuration; and
transforming the proximal member from the retracted configuration to the deployed configuration, thereby sealing the perforation between the distal and the proximal members.

13. The method of claim 12, further comprising positioning the proximal member adjacent the perforation.

14. The method of claim 13, wherein the positioning step is completed prior to transforming the proximal member.

15. The method of claim 13, wherein the positioning step is completed after transforming the proximal member.

16. The method of claim 12, wherein the body further comprises an intermediate member, a portion of which surrounds the inner member.

17. The method of claim 16, wherein transitioning the distal member from the retracted to the deployed configuration comprises retracting the inner member to cause the distal member to compress against the intermediate member, thereby causing compression and radial expansion of the distal member.

18. The method of claim 12, wherein the proximal member is an expandable balloon.

19. The method of claim 18, wherein transitioning the proximal member from the retracted to the deployed configuration comprises inflating the expandable balloon.

20. The method of claim 12, wherein the tissue is a blood vessel.

21. The method of claim 12, further comprising detaching the distal member from the body.

22. The method according to claim 12, further comprising extracting the device from the perforation by:
transforming the proximal member from the deployed configuration to the retracted configuration;
transforming the distal member from the deployed configuration to the retracted configuration; and
removing the device from the perforation.
